(19) United States
(12) Patent Application Publication
(10) Pub. No.: US 2006/0263906 A1
(43) Pub. Date: Nov. 23, 2006

(54) Tri-functional nanospheres

(75) Inventors: Dai-Wen Pang, Wuhan (CN); Hai-Yan Xie, Wuhan (CN); Guo-Ping Wang, Wuhan (CN); Zhi-Ling Zhang, Wuhan (CN); Er-Qun Song, Wuhan (CN); Yun-Bo Shi, Bethesda, MD (US)

Correspondence Address: Birch, Stewart, Kolasch & Birch, LLP, 8110 Gatehouse Rd, Suite 500 East, P.O. Box 747, Falls Church, VA, 22040-0747, US

(73) Assignees: Wuhan University; The Government of the U.S as Represented By the Secretary of the Dept of Health and Human Services

(21) Appl. No.: 11/135,380

(22) Filed: May 24, 2005

(30) Foreign Application Priority Data
May 23, 2005 (CN) 200510079227.0

(51) Int. Cl. G01N 33/551

(52) U.S. Cl. 436/524; 977/900

(57) ABSTRACT

Trifunctional nanoparticles have excellent fluorescence, magnetism, and cell recognition, which can be easily manipulated, tracked, and conveniently used to capture target cells. The surface-immobilized molecules of the TFNs might be optionally changed on demand for the purposes of bioanalysis, biomedical imaging, diagnosis, and the combinatorial screening of drugs. The nanoparticle is formed from a mesoporous polymer; a magnetic material adhering to the mesoporous polymer; a fluorescent dye adhering to the mesoporous polymer; and a biomaterial coupled to the mesoporous polymer, where the mesoporous polymer has been treated with hydrazine, and the biomaterial has been treated with an oxidizing agent.

This non-provisional application claims the benefit under 35 U.S.C. §119 of a Chinese Application having attorney's reference number IIC051309, filed on May 23, 2005, which is hereby incorporated in its entirety by reference.

### BACKGROUND OF THE INVENTION

1. Field of the Invention

The invention relates to tri-functional or multi-functional nanoparticles obtained by binding a biomaterial to a fluorescent-magnetic bifunctional nanoparticle (BFN).

2. Description of the Related Art

Fluorescent labeling and magnetic separation are two important bio-techniques. Materials with the combined function of these two properties have many applications in biomedical science.

Nanospheres are becoming the materials of choice for a rapidly increasing number of pharmaceutical applications and in biomedical research. In the related art, several methods have been developed using quantum dots (QDs) and magnetic nanoparticles, and for encapsulating both particles in polymer microcapsules.

However, these related art technologies are predominantly dependent on core-shell type technologies. Typically, a magnetic material such as magnetite or a fluorescent particle such as a QD is used as a core. Such a core-shell structure is disadvantageous, especially for fluorescence applications, in that the shell tends to absorb either or both of the excitation and emission light, thus dimming the fluorescent signal. In some embodiments of the related art, a fluorescent molecule is incorporated into the material of the shell. In these instances, the fluorescence signal can be dimmed by transfer of energy from the excited fluorophore to the surrounding solid matrix.

### BRIEF DESCRIPTION OF THE DRAWINGS

The accompanying drawings, which are included to provide a further understanding of the invention and are incorporated in and constitute a part of this application, illustrate embodiments of the invention and together with the description serve to explain the principle of the invention.

In the drawings:

FIG. 1A-B shows an X-ray diffraction (XRD) pattern indexed onto a face-centered cubic cell with a=0.835 nm and a transmission electron microscope (TEM) image of nano-γ-Fe₂O₃.

FIG. 2 shows a TEM image of nanospheres embedded with both CdSe/ZnS QDs and nano-γ-Fe₂O₃.

FIG. 3A-3D shows HRTEM High Resolution Transmission Electron Microscope (HRTEM) images of CdSe/ZnS QDs and γ-Fe₂O₃ nanoparticles embedded in hydrazine-treated styrene/acrylamide copolymer (H₂N-St-AA).

FIG. 4A-B shows bright field and fluorescent microscopic images of bifunctional nanoparticles (BFNs).

FIG. 5 shows a schematic representation of the fabrication of a trifunctional nanoparticle (TFN) and its capturing of a cancer cell.

FIG. 6A-I shows fluorescent microscopic images of trifunctional nanoparticles (TFNs) with folic acid as the biomaterial, and binding of the TFNs to cancer cells bearing folic acid receptors.

FIG. 7 shows a diagram of schemes for the preparation of tri-functional nanoparticles.

FIG. 8A-8D shows photomicrographs of trifunctional anti-rabbit IgG-nanoparticles bound to rabbit anti-human IgG-FITC.

FIG. 9A-9D shows a photomicrograph of tri-functional biotin-nanoparticles bound to streptavidin-phycoerythrin.

### DESCRIPTION OF THE INVENTION

The development of bifunctional nanoparticles with higher structural stability than has been achieved in the prior art, and also demonstrating specific binding to cells, proteins or other particles is highly desired. Such specific binding can be achieved by coupling of a biomolecule providing specific recognition of a receptor or the like. Also, coupling of a biomaterial to the nanoparticle becomes important as more specific applications, e.g., cancer cell capture, are desired.

The invention is directed to producing a multi-functional nanoparticle or “nanosphere” that substantially obviates one or more problems due to limitations and disadvantages of the related art. Any particular embodiment of the invention might not solve every problem of the related art described above.

The invention, in part, pertains to a nanoparticle composed from a mesoporous polymer, a magnetic material adhering to the mesoporous polymer, a fluorescent dye adhering to the mesoporous polymer, and one or more biomaterials coupled to the mesoporous polymer. The nanoparticle can have a spherical shape or may be agglomerated. Nanoparticles of the invention may form into aggregates in some instances, but generally are dispersed when suspended in a liquid.

In instances where the biofunctionalized nanoparticles (“trifunctional” or “multifunctional” nanoparticles) of the invention are labeled by a single biomaterial, the nanoparticles may specifically bind to a cell, or a protein or any other moiety that to which the biomaterial specifically binds. For instance, the biomaterial may be a small molecule ligand that is specifically bound by a cell surface receptor.

In one aspect, the invention provides more than one active biomaterials bound to a bi-functional nanoparticle. Potential applications of multi-functional nanoparticles of the invention in which two bioagents are coupled to a single bifunctional nanoparticle include using one bioagent to target a macromolecular or a cell and using the second one to alter the function/properties of the macromolecule or cell, e.g., using a protein to target a cell and using a toxin or cell death protein to kill the targeted cell, or using a chemical or protein to target a protein within a complex and another one to alter the function of a different component of the complex.

In one embodiment, for coupling of the biomaterial to the nanoparticle, the mesoporous polymer has been treated with hydrazine, and the biomaterial has been treated with an oxidizing agent.

The biomaterial can be a ligand such as a peptide or protein. Protein embodiments may encompass peptides and preferred protein embodiments of the biomaterial are glycoproteins. The biomaterial can also be a small molecule. The biomaterial can be an antibody or a ligand specific for a desired cellular receptor. The biomaterial can also be a carbohydrate. Selection of the biomaterial from such materials as IgG, avidin, biotin or streptavidin allows complexation of the TFNs of the invention to a wide variety of substances that may be conjugated to binding partners for these molecules.

For coupling of the biomaterial to the nanoparticle, the mesoporous polymer has been treated with hydrazine, and the biomaterial has been treated with an oxidizing agent. The biomaterial can be a ligand such as a peptide or a glycoprotein. The biomaterial can also be a small molecule. The biomaterial can be an antibody or a ligand specific for a desired cellular reception. The biomaterial can also be a carbohydrate ligand. Typically, the biomaterial can be selected from but is not restricted to such materials as IgG, avidin, biotin or streptavidin.

The magnetic material can be Fe₂O₃.

The fluorescent dye can be CdSe or CdSe/ZnS quantum dots.

The polymer can be hydrazinized styrene/acrylamide (H₂N-St-Aam).

In the invention, the biomaterial can be coupled to the nanoparticle with the following structure: 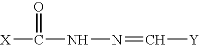
where X is the nanoparticle and Y is the biomaterial. Y can be a ligand, a peptide, a protein or a glycoprotein. Examples of such biomaterials are an antibody, avidin or streptavidin. Avidin is a glycoprotein. Streptavidin is not a glycoprotein. That is, the above linkage is suitable for use with glycoproteins, including antibodies and avidin and other proteins such as streptavidin, which can be oxidized to generate and aldehyde group, and also for other biomaterials that include an aldehyde group. If the biomaterial Y is biotin, then the nanoparticle can have the following structure: 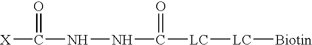
where X is the nanoparticle and LC is —C═O(CH₂)₃—NH—. In the invention, the Sulfo-NHS-LC-LC-Biotin (Sulfosuccinimidyl-6′-(biotinamido)-6-hexanamido hexanoate) has the following structure:

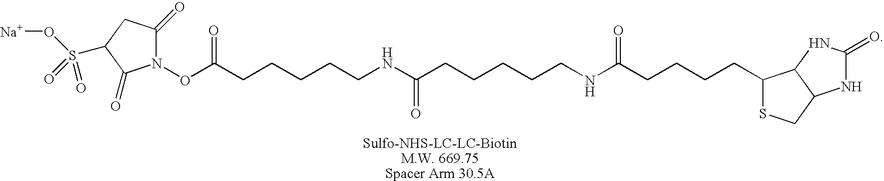

The invention, in part, pertains to a multifunctional nanoparticle, having the following structure: 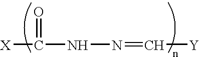
where n≧1; x is a mesoporous nanoparticle formed from a mesoporous polymer, a magnetic material adhering to the mesoporous polymer and a fluorescent dye adhering to the mesoporous polymer; and Y is a protein.

The invention, in part, pertains to a method for forming a multifunctional nanoparticle that includes providing a mesoporous polymer nanoparticle, the nanoparticle having a magnetic material adhering to the mesoporous polymer, a fluorescent dye adhering to the mesoporous polymer; treating the nanoparticle with hydrazine; oxidizing a biomaterial; and coupling the oxidized biomaterial to the nanoparticle. The biomaterial can be oxidized using sodium metaperiodate. Other oxidants can be used, including but not being restricted to potassium metaperiodate or any other metaperiodate.

Any protein can be coupled to nanoparticles. Glycoproteins are most easily coupled, as they can be oxidized to generate an active aldehyde group. Other proteins can be coupled via their —COOH group(s) but with lower efficiency. However, other means known in the art, such as di-imide reagents, e.g. carbodiimide can be used to couple proteins lacking sugars to the nanoparticles.

In preferred embodiments, the biomaterial can be IgG, avidin, biotin or streptavidin. The IgG, avidin, biotin or streptavidin may be further conjugated to other molecules, which in turn may serve as ligands for desired receptors or as functional molecules such as toxins.

The nanoparticles of the invention preferentially have no magnetic core. Rather, magnetism is imparted to the nanoparticle of the invention by association of the polymer nanoparticle with smaller magnetic nanoparticles.

The invention, in part, pertains to a multifunctional nanoparticle having the following structure:

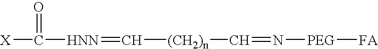

where n≧1; X is a mesoporous nanoparticle formed from a mesoporous polymer, a magnetic material adhering to the mesoporous polymer and a fluorescent dye adhering to the mesoporous polymer; PEG is polyethoxyethylene; and FA is folic acid. Preferably, n is 3.

The nanoparticles of the invention can be derivatized using small molecule ligands other than the exemplified folic acid and biotin. In fact, any bioagent with reactive groups such as —COOH, —CHO, —NH₂ and —SH etc. can be coupled to the surface. Those with —COOH and —CHO can be coupled directly without a crosslinker. Biotin can be obtained commercially that is already modified or activated as to be ready for direct coupling.

The invention also relates to methods for labelling and collecting cells or moieties recognized by the ligands attached to the TFNs of the invention. For instance, the invention provides a method for separating cells in which cells bearing a desired receptor are introduced to a multi-functional nanoparticle in which Y is a ligand that specifically binds to the desired receptor, thus labeling the cells with multifunctional nanoparticles. The cells having multifunctional nanoparticles bound on their surface are then introduced into a magnetic field, which immobilizes the cells, for example at the bottom of a test tube or in the bottom of a multiwell plate. Then cells that are not labeled by the multifunctional nanoparticles can be washed away and then the magnetic field can be removed and the cells collected.

The above method can be extended to separating cells of bearing different kinds of receptors from a sample using the fluorescence function of the multifunctional nanoparticles. In this method, a sample of cells bearing a plurality of desired receptors is introduced to a mixture of multi-functional nanoparticles of different kinds, in which each kind of multi-functional nanoparticle has a different ligand Y that specifically binds to a desired surface receptor on at least one cell in the sample and further in which each ligand Y is paired with a fluorescent dye of a particular color, to obtain cells bound with multifunctional nanoparticles of different colors. The various colors of the multifunctional nanoparticles labeling the cells will reflect the different receptors for the different ligands Y present on the cells. The labelled cells are then introduced to a magnetic field to immobilize them. Then unlabeled cells are removed and the labeled cells are collected. The collected cells are then sorted according to the color of the multifunctional nanoparticles on their surface, for example by fluorescence activated cell sorting. In this manner cells having different desired surface markers can be separated and collected or counted.

The invention, in part, pertains to a method for isolating and/or detecting biomolecules, which includes contacting a biomixture containing a biomolecule bearing a desired interacting site with the inventive multi-functional nanoparticle described above, in which Y is composed of a ligand that specifically binds to a desired receptor or other binding partner to obtain biomolecules bound with multifunctional nanoparticles; introducing the biomolecules bound with multifunctional nanoparticles into a magnetic field, thereby immobilizing the biomolecules and associated molecules; removing any molecules not bound with the multifunctional nanoparticles; removing the magnetic field from the biomolecules bound with multifunctional nanoparticles; and collecting the bound biomolecules. The method can further include purifying the bound biomolecules and associated molecules via the fluorescence of the biomolecules bound with the multifunctional nanoparticles. The step of further purifying the bound biomolecules occurs either before or after the step of collecting the bound biomolecules, or after the biomolecules and associated molecules ar immobilized. Also, the biomolecule can be a protein, but is not restricted to a protein.

Reference will now be made in detail to the preferred embodiments of the invention, examples of which are illustrated in the accompanying drawings. The figures and preferred embodiments should not be considered as limiting of the invention; the scope of the invention is defined only by the claims following.

Bi-functional nanoparticles (BFNs) were prepared by embedding fluorescent CdSe/ZnS quantum dots (QDs) and magnetic nano-γ-Fe₂O₃ into hydrazine-treated styrene/acrylamide (H₂N-St-AAm) copolymer nanospheres simultaneously. In order to ensure that both the QDs and magnetic nanoparticles are embedded, the particles must be small and well dispersed in a single solution such as 5:95 (V/V) chloroform:butanol. The invention is not restricted to chloroform:butanol, and any appropriate solvent or solvent mixture can be used.

Nano-γ-Fe₂O₃ was prepared through the high temperature method reported by S. Sun et al., J. Am. Chem. Soc. (2002) vol. 124, pp. 8204-8205.

Monodisperse CdSe QDs were obtained, as described by L. H. Qu et al. J. Am. Chem. Soc. (2002), vol. 124, pp. 2049-2055.

Styrene/acrylamide nanoparticles were obtained as described in X. Zhao et al., J. Med. Coll. PLA (1997) vol. 12, pp. 62-65. The diameter of microspheres was 3±0.05 μm and the content of carboxyl groups on the surface was 190.5 μmol/g (dry solids).

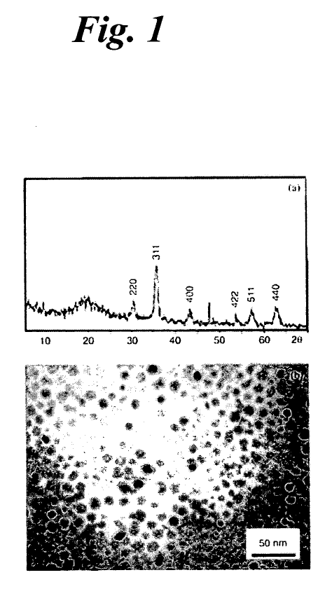

FIG. 1A shows an X-ray diffraction (XRD) analysis of the iron oxide, which indicates that the crystal structure of the product was cubic γ-Fe₂O₃ rather than Fe₃O₄. This product was obtained because the synthesis was carried out in air and in the absence of 1,2-hexadecanediol, which may reduce iron cations. Transmission electron microscopy (TEM) results shown in FIG. 1B indicate that the iron oxide particles have an average particle size smaller than 20 nm with a narrow size distribution and are well dispersed.

St-AAm copolymer nanoparticles were synthesized from styrene and acrylamide by a modified method of emulsifier-free polymerization, as described in X. Zhao et al., J. Med. Coll. PLA (1997), vol. 12, pp. 62-65. The size of the spheres can be changed from 50 to 500 nm by adjusting the concentration of raw materials and the dosage of the reaction initiator. When the concentration of the reaction initiator is fixed, and the concentration of raw materials increases, then the sphere size increases. If the amount of reaction initiator increases, and the concentration of raw materials is fixed, then the sphere size decreases. Scanning electron microscopy (SEM) images imply that the surface of the spheres is mesoporous, thus providing an entry route for nanoparticles. As the polymerization is emulsifier-free, the surface is relatively clean and convenient for conjugating with other molecules.

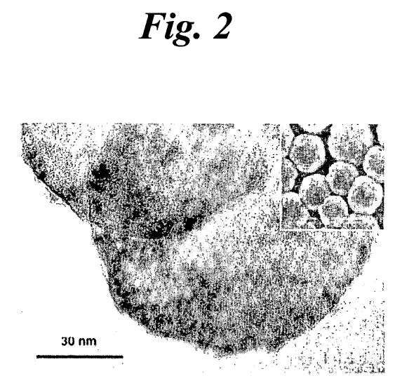

Without being bound by any theory of the invention, the inventors believe that the hydrophilic groups of the polymer tend to be located towards the outer surface of the nanospheres, while the hydrophobic moieties are found at the interior, leading to the formation of hydrophobic hollow cavities, since the nanospheres are synthesized in an aqueous solution. Both hydrophobic QDs (3-6 nm) and nano-γ-Fe₂O₃ (5-20 nm) were embedded in a weakly polar organic solvent. An example of a weakly polar organic solvent is butanol and solvents that are close to butanol in terms of polarity. At the same time 15% (by weight) or less of chloroform is added to the solvent in order to well disperse both the hydrophobic QDs and the nano-γ-Fe₂O₃. Preferably from 5 to 15% of chloroform or a similar non-polar solvent is used. FIG. 2 shows a transmission electron microscope (TEM) image of nanospheres embedded with both CdSe/ZnS and nano-γ-Fe₂O₃, and the inset shows a scanning electron microscope (SEM) image of H₂N-St-Aam nanospheres. As shown in FIG. 2, the particles are widely distributed inside the nanospheres with a relatively clean surface.

Energy-dispersive X-ray (EDX) microanalysis was carried out to confirm the distributions of these particles. When the BFNs were dispersed in a polar aqueous solution, no detectable leakage of the embedded hydrophobic particles from the hydrophobic cavities were observed, even after continuous ultrasonication for one week. This result indicates that the hydrophobic particles were embedded inside the nanoparticles as desired.

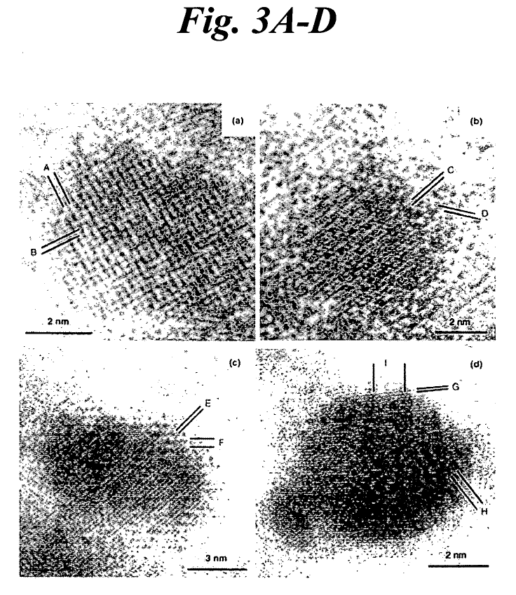

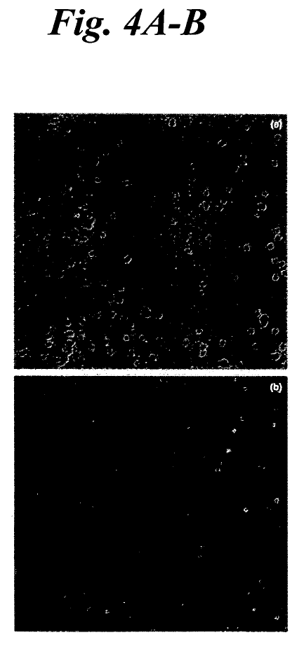

FIG. 3 shows high-resolution TEM (HRTEM) images of some individual QDs and γ-Fe₂O₃ nanoparticles embedded in the H₂N-St-AAm copolymer. FIG. 3A is an image of a QD (CdSe/ZnS QD; A: (102 direction), d=0.25 nm; B: (111 direction), d=0.21 nm. The interplane angle is 92°). The crystal data can be almost fit to the structure of wurtzite CdSe with the unit cell parameters a=0.4299 and c=0.7010 nm. FIG. 3B is another QD viewed down a different zone axis (CdSe/ZnS QD; C: (111), d=0.21 nm; D: (201), d=0.17 nm. The interplane angle is 57°.) FIG. 3C shows a QD and a γ-Fe₂O₃ particle, with different sizes overlapping each other. In FIG. 3C, atomic planes of E of the small crystallite can be indexed as CdSe(III) with d=0.21 nm and F of the large particle indexed as cubic γ-Fe₂O₃(200) with d=0.41 nm. Another image of a γ-Fe₂O₃ nanoparticle is shown in FIG. 3D, with the fringes G indexed to (222) with d=0.24 nm. Large d spacings corresponding to some superstructures are also observed. The magnetic response of the BFNs is adequate, and the BFNs dispersed in solvent could be manipulated easily. It only takes tens of seconds to collect and manipulate the BFNs and later the nanosphere-captured cells. The BFNs were also found to be of good dispersivity as shown in FIG. 4A (bright field microscope) and FIG. 4B (fluorescence microscope).

Relative UV/Vis absorption intensities of γ-Fe₂O₃ nanoparticles are different at different wavelengths. On the other hand, the fluorescence emission wavelength of QDs depends on their particle diameter. The smaller the particles are, the shorter the wavelength of the fluorescence. Therefore, the influence of γ-Fe₂O₃ on the fluorescence intensity by absorption of the fluorescent light will change with the particle diameter of the QDs and the relative dosages of both kinds of the nanoparticles. Although this interactoin always exists to some extent, the fluorescence of QDs is strong enough to be directly observed with a fluorescence microscope in all cases (FIG. 4B).

FIG. 4 shows the homogeneous nature of the fluorescing particles. Hence, the dosage ratio of γ-Fe₂O₃ to the QDs and the concentration of the particles can be quite flexible. If more fluorescence is required or desired, more QDs will be included. If less fluorescence is required or desired, fewer QDs will be included.

Folic acid is one ligand that may be used to obtain tri-functional nanoparticles (TFNs) of the invention. TFNs may be obtained by modifying the surface of the BFNs with folic acid (FA), which is a vitamin required for one-carbon transfer reactions along several metabolic pathways, and folic acid is essential for the biosynthesis of nucleotide bases. Folic acid is based on 4-[(pteridin-6-ylmethyl)amino]benzoic acid (pteroic acid), which has the IUPAC nomenclature 4-{[(2-amino-3,4-dihydro-4-oxopteridin-6-yl)methyl]amino}benzoic acid. The compounds in which pteroic acid is conjugated with one or more molecules of L-glutamate are named pteroylglutamate, pteroyldiglutamate, etc. Folate and folic acid are the preferred synonyms for pteroylglutamate and pteroylglutamic acid, respectively.

The FA receptor (FR) is a glycoprotein that is found to be vastly over-expressed in a wide variety of human tumors, especially in epithelial cancer cells. The binding affinity between FA and FR is very high, with a dissociation constant K_{d} of about 10⁻⁹ to 10⁻¹⁰ M.

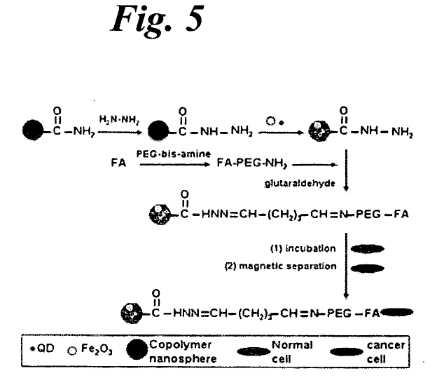

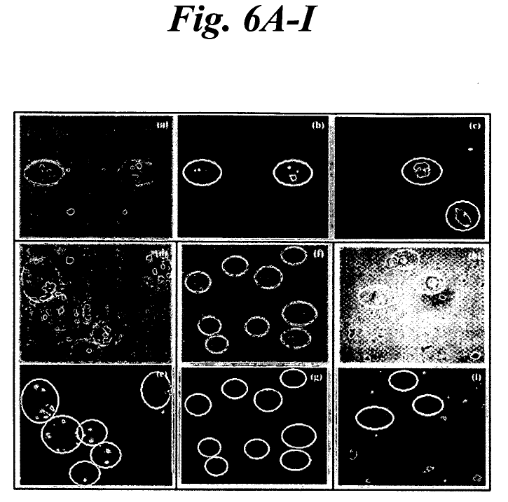

FIG. 5 depicts a scheme for the fabrication of a TFN and how it captures a cancer cell. FIG. 6A-C shows MCF-7 cells incubated with the TFNs for 3 h (B) and 6 h (C), with a maximum emission wavelength of 540 nm for the QDs (A) bright field; (B), (C) fluorescence (FL)). FIG. 6D-E shows Hela cells incubated with the TFNs for 4 h, with a maximum emission wavelength of 595 nm for the QDs (D) bright field; E) fluorescence. FIG. 6F-G shows the control experiment, with MRC-5 cells incubated with the TFNs for 6 h, with a maximum emission wavelength of 595 nm for the QDs (F) bright field; G) fluorescence). The supernatant after magnetic separation was taken for observation. FIG. 6H-I shows the control experiment, with MCF-7 cells incubated with BFNs for 6 h, with a maximum emission wavelength of 610 nm for the QDs (H) bright field; I) fluorescence; without magnetic separation.

A hydrazination reaction was utilized to include a —NH— moiety to enhance the reactivity of the NH₂ group on the surface. Polyoxyethylene-bis-amine (PEG-bis-amine) was used as a spacer. PEG is also referred to as polyethylene glycol. Glutaraldehyde was used to activate the H₂N-St-AAm nanospheres for further attachment of FA-PEG-NH₂. Glutaraldeheyde (HCO(CH₂)₃OCH) is the preferred activating agent. However, the invention is not restricted to glutaraldehyde, and any appropriate dialdehyde or formaldehyde can be used. Also, molecules with multiple aldehyde groups can be used.

TFNs with FA ligands can capture Hela and MCF-7 cancer cells after 3-4 h incubation. As the incubation time was increased, more TFNs were found on the cell surface (FIG. 6A-6E). Control experiments showed that even if the TFNs were incubated with MRC-5 cells (a type of FR-deficient normal human cell) for 6 h under the same conditions, no capture occurred (FIGS. 6F and 6G). For FA-free BFNs, there was no obvious interaction between the BFNs and MCF-7 cells even after 6 h incubation. Therefore, no fluorescence was observed on the cell surface and BFNs aggregated in the cell culture media (FIGS. 6H and 6I). The nonbinding BFNs in FIG. 6H and FIG. 6I were intentionally left without washing, so as to indicate that they could not interact with the cells when coexisting in culture medium. After washing, no fluorescent BFNs can be observed on the cells. All of these results indicate that the TFNs can capture the cancer cells through the specific recognition interaction between FA and FR. On the other hand, the nonspecific adsorption can be almost eliminated, which can not be done in the case of using nanoparticles alone.

The invention also permits the utilization of novel binding mechanisms to attach biomaterials to bifunctional nanoparticles. Combining the fluorescent and magnetic property into a single nanosphere greatly increases its application potential in the biomedical and biopharmaceutical fields. The invention includes the fabrication of fluorescent-magnetic bifunctional nanospheres by co-embedding quantum dots and nano-γ-Fe₂O₃ into poly(styrene/acrylamide) copolymer nanospheres. The subsequent biofunctionalization of these nanospheres (100-150 nm in diameter) with immunoglobulin G (IgG), avidin, streptavidin or biotin generates trifunctional nanospheres with wide range of biomedical applications.

The exemplary fluorescent probe material used in the invention was CdSe. However, any suitable semiconductor material can be used. These semiconductor materials include but are not restricted to CdTe, CuInSe₂, CdS, InGa, InAs, CdSe/ZnS, PbSe, etc.

Also, the magnetic material is not restricted to Fe₂O₃. Other suitable magnetic materials include, but are not restricted to Co, Co alloys, Co ferrite, Co nitride, Co oxide, CoPd, CoPt, Fe alloys, FeAu, FeCr, FeN, Fe₃O₄, FePd, FePt, FeZrNbB, MnN, NdFeB, NdFeBNdCu, Ni and Ni alloys.

The preferred polymer used as the basis of the inventive trifunctional nanoparticles is hydrazine-treated styrene/acrylamide. However, the invention is not restricted to this polymer system, and any appropriate polymer can be used. The polymer systems include, but are not restricted to, polymers and copolymers of polystyrene, polycarbonate, polymethylmethacrylate, polymethylacrylates, other suitable acrylic or methacrylic systems, and polyethylene.

For example, styrene and acrylamide can be used to synthesize the styrene/acrylamide copolymer nanospheres. Water (150 ml), acrylamide (5 g) and NaCl (0.3 g) can be mixed and heated to 70° C. under a still N₂ atmosphere for at least 15 min, styrene (22 ml) was added for about 10 min. Then, 0.1 g of potassium persulfate dissolved in 20 ml of water is introduced while stirring. The polymerization is carried out at 70° C. for 7 h in a still N₂ atmosphere. Finally, the copolymer nanospheres are separated from the reaction solution and could be modified with hydrazine through its surface amide functional groups so as to produce a reactive nanosphere surface. If other polymeric materials are used, the details of the reaction conditions may vary.

### EXAMPLES

To prepare CdSe/ZnS QDs, monodisperse CdSe QDs were first obtained, as described by L. H. Qu et al. J. Am. Chem. Soc., (2002) vol. 124, pp. 2049-2055. The precursors were prepared from hexamethyldisilathiane ((TMS)₂S) and zinc acetylacetonate (Zn(ac)₂) were added dropwise into a freshly prepared CdSe solution at 200° C. Nano-γ-Fe₂O₃ was prepared from the reaction of ferric acetylacetonate, hexadecylamine (HDA), and oleic acid. St-AAm copolymer nanospheres were fabricated by polymerization of St (styrene) and AAm (acrylamide) in an aqueous solution. H₂N-St-AAm was prepared by a hydrothermal treatment of St-AAm with hydrazine. The hydrothermal treatment was treatment of St-AAm with hydrazine in warm water at about 45° C.

The fabrication of BFNs was achieved by swelling the H₂N-St-AAm in a chloroform/butanol solvent (5:95 v/v), and a controlled amount of CdSe/ZnS QDs and nano-γ-Fe₂O₃. The mixture was then ultrasonicated for 30 min, centrifuged, and washed with butanol three times. Specimen characterization was performed on an Acc. V Spot Maqn SEM operated at 20 kV, a JEOL-JEM 2010 TEM operated at 200 kV, an Oxford Link ISIS system for EDX, and a D/max-RC diffractometer.

Folic Acid Coupling

For the synthesis of FA-PEG-NH₂, FA (90 mg) in anhydrous dimethyl sulfoxide (4 mL) and triethylamine (1 mL) was reacted with N-hydroxysuccinimide (NHS; 50 mg) in the presence of dicyclohexylcarbodiimide (DCC; 51 mg) at room temperature. The reaction mixture was stirred for 4 h in darkness, followed by the addition of PEG-bis-amine (MW-3350, 670 mg). The resulting mixture was then stirred overnight. Dicyclohexylurea was removed by filtration. Thin-layer chromatography on silica gel containing both plaster of Paris and fluorophores (silica gel GF)(75:36:6 chloroform/methanol/water) showed a new spot (R_{f} ˜0.56) due to the formation of the product FA-PEG-NH₂. The supernatant was dialyzed against saline (NaHCO₃, pH 8.0, 50 mm) and water, and was finally lyophilized. The hydrazinized nanospheres were coupled to the FA-PEG-NH₂ by glutaraldehyde crosslinking.

Targeting of FA-Coupled TFNs to Cancer Cells

For demonstrating the use of FA-coupled TFNs for cancer-cell targeting, Hela cells (a human cervical carcinoma cell line), MCF-7 cells (a human breast cancer cell line), and MRC-5 cells (a diploid human lung fibroblast cell line) were routinely cultured at 37° C. in flasks containing FA-free RPMI-1640 medium, supplemented with 10% fetal calf serum (which was the sole source of FA) in a humidified atmosphere with 5% CO₂ (in air). To perform cell capturing by the biofunctional nanospheres (FA-PEG-BFN), the cells were first cultured in six-well plastic dishes for 24 h (cancer cells) and 48 h (normal cells), and then reseeded culture medium having dispersed in it the TFNs prepared as above. The cells were incubated at 37° C. for a fixed time (from 3 to 6 h; see FIG. 5). The cells were washed several times with phosphate buffered saline (pH 7.4 PBS) to remove nonspecifically adsorbed FA-PEG-BFN nanospheres, detached using trypsin-EDTA solution, resuspended in culture medium, and magnetically separated. The supernatant was discarded and the magnetically separated cells were suspended in pH 7.4 phosphate buffer solution (PBS). The suspension (−10 mL) was dropped onto a slide for fluorescence microscopy observation.

Immunoglobulin G Coupling

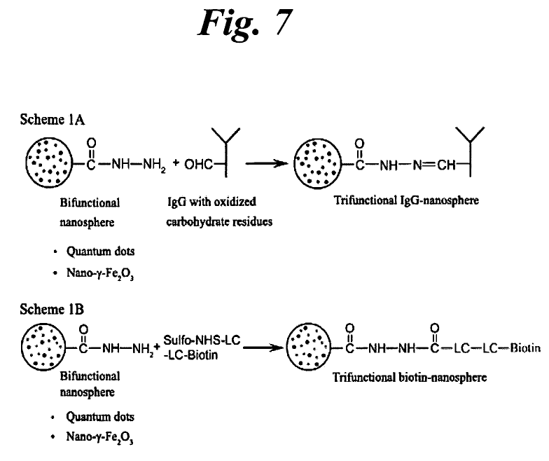

Goat anti-rabbit IgG (0.4 mL, 5 mg/mL, Sigma) was oxidized to create active aldehydes in its Fc fragment with sodium metaperiodate (0.1 mL, 50 mmol/L in 0.1 M pH6.8 PBS, Sigma) in an amber vial for 30 min at room temperature with constant shaking. Also, potassium periodate or any other periodate will preferentially produce CHO groups and may be substituted for sodium metaperiodate. The reaction was stopped and unreacted sodium metaperiodate was removed by passing the mixture through a desalting column (PD10, Amersham Biosciences). Hydrazide-containing bifunctional nanospheres embedded with orange-red quantum dots were completely resuspended by sonication for a few min after third wash with PBS. The suspension of hydrazide-containing bifunctional nanospheres (0.5 mL, 20 mg/mL in PBS) and the oxidized antibody (0.5 mL, ca. 4 mg/mL in 0.1 M pH6.8 PBS) were mixed and incubated with constant shaking for at least 6 h at room temperature (FIG. 7, Scheme 1A), and subsequently washed ten times with PBS. As a result, fluorescent-magnetic-biotargeting trifunctional IgG-nanospheres were obtained.

There size of the QDs correlates to the fluorescence color. The factors affecting the size of the QDs include the concentration of raw materials and the termperature and time of the reaction. A detailed explanation of control of QD color can be found in L. Qu et al., (2002) J. Am. Chem. Soc., vol. 124, pp. 2049-2055 and in L. Qu et al., (2001) Nano Letters, vol. 1, pp. 333-337. QDs of different colors are commercially available from Jiayuan Quantum Dots Co., Ltd in China and from Quantum Dot Corporation in the USA.

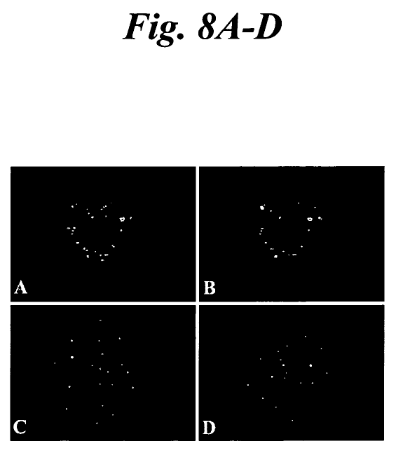

To characterize the bioactivity of goat anti-rabbit IgG on their surface, the trifunctional nanospheres (0.2 mL, in 0.1 M pH7.2 PBS) as above were incubated with rabbit anti-human IgG-FITC (10 μL, 3.0 g/L, Beijing Zhongshan Golden Bridge Biotech. Co. Ltd.) for 30 min at 4° C. with gentle shaking, followed by washing ten times with PBS to remove the unbound rabbit anti-human IgG-FITC. Microscopic analysis of the fluoroceinyl isothiocyanate (FITC) fluorescence clearly demonstrated the binding of rabbit anti-human IgG to the goat anti-rabbit IgG on the surface of the nanospheres (coincident green and red fluorescence shown in FIGS. 8A and 8B), indicating that the activity of the goat anti-rabbit IgG was preserved during the coupling process. On the other hand, no fluoroceinyl isothiocyanate (FITC) fluorescence was detected on the nanospheres when rabbit anti-human IgG-FITC was incubated with the nanospheres coupled with non-oxidized goat anti-rabbit IgG antibody (FIG. 8C), or when bifunctional nanospheres was incubated with rabbit anti-human IgG-FITC (FIG. 8D). These results demonstrated that the trifunctional nanospheres specifically recognized rabbit anti-human IgG through their surface goat anti-rabbit IgG and that covalent coupling was needed to generate such trifunctional nanospheres.

Avidin Coupling

To investigate the versatility of biofunctionalization of bifunctional nanospheres, trifunctional nanospheres were prepared using avidin. Avidin was coupled to the bifunctional nanospheres as described above for goat anti-rabbit IgG. When these avidin-nanospheres were incubated for 1 h with biotin-FITC in PBS, followed by thorough washing with PBS to remove excess biotin-FITC.

Biotin capture by the avidin on the surface of the nanospheres was confirmed by fluorescence microscopy, while no capture occurred when bifunctional nanospheres without avidin coupling was incubated with biotin-FITC, or when biotin-FITC was incubated with the nanospheres having incubated with non-oxidized avidin, again demonstrating the specificity and bioactivity of the trifunctional nanospheres.

Biotin Coupling

Biotin was coupled to bifunctional nanospheres. Sulfo-NHS-LC-LC-biotin (4.8 mg, Pierce) was directly added to the suspension of hydrazide-containing bifunctional nanospheres (0.5 mL, 20 mg/mL in PBS) embedded with green quantum dots, followed by a 3 h reaction with shaking at room temperature and subsequent washing for ten times with PBS to produce trifunctional nanospheres with surface biotin (FIG. 7, Scheme 1B).

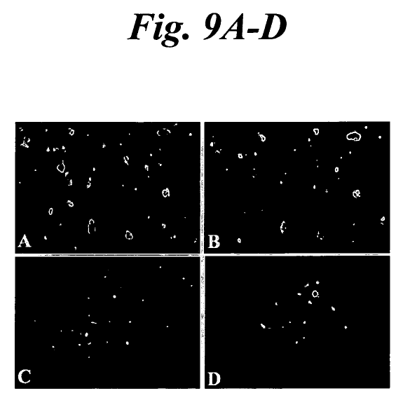

To assess the bioactivity of biotin on their surface, streptavidin-phycoerythrin (10 uL, Sigma) was added to trifunctional biotin-nanospheres. (0.2 mL) and incubated for 1 h at room temperature with gentle shaking, followed by thorough washing with PBS. Analysis of phycoerythrin fluorescence demonstrated streptavidin-binding to the surface of the biotin-nanospheres (coincident red and green fluorescence in FIGS. 9A and 9B), while the binding did not take place with nanospheres coupled with unmodified biotin (FIG. 9C) or with bifunctional nanospheres without biotin coupling and streptavidin-phycoerythrin (FIG. 9D).

In conclusion, a simple and convenient strategy was used to fabricate novel trifunctional nanospheres with excellent fluorescence, magnetism, and cell recognition, which can be easily manipulated, tracked, and conveniently used to capture target cells. Furthermore, the surface-immobilized molecules of the TFNs might be optionally changed on demand for the purposes of bioanalysis, biomedical imaging, diagnosis, and the combinatorial screening of drugs.

It will be apparent to those skilled in the art that various modifications and variations can be made in the invention as described hereinabove without departing from the spirit or scope of the invention. It is intended that the invention covers the modifications and variations of this invention provided they come within the scope of the appended claims and their equivalents.

Various articles of the scientific periodical literature are cited in this application. Each such article is hereby incorporated by reference in its entirety and for all purposes by such citation.

1. A nanoparticle comprising:
a mesoporous polymer;
a magnetic material adhering to the mesoporous polymer;
a fluorescent dye adhering to the mesoporous polymer; and
a biomaterial coupled to the mesoporous polymer, wherein the mesoporous polymer has been treated with hydrazine, and the biomaterial has been treated with an oxidizing agent.

2. The nanoparticle according to claim 1, wherein the biomaterial is selected from the group consisting of IgG, avidin, biotin and streptavidin.

3. The nanoparticle according to claim 1, wherein the magnetic material comprises Fe₂O₃.

4. The nanoparticle according to claim 1, wherein the fluorescent dye comprises CdSe or CdSe/ZnS quantum dots.

5. The nanoparticle according to claim 1, wherein the polymer comprises hydrazine-treated styrene/acrylamide (H₂N-St-Aam).

6. A method for forming a multifunctional nanoparticle, comprising:
providing a mesoporous polymer nanoparticle, the nanoparticle having a magnetic material adhering to the mesoporous polymer, a fluorescent dye adhering to the mesoporous polymer;
treating the nanoparticle with hydrazine;
oxidizing a biomaterial; and
coupling the oxidized biomaterial to the nanoparticle.

7. The method according to claim 6, wherein the biomaterial is oxidized using sodium metaperiodate.

8. The method according to claim 6, wherein the oxidized biomaterial has an active aldehyde group.

9. The method according to claim 6, wherein the biomaterial is selected from the group consisting of IgG, avidin, biotin and streptavidin.

10. The method according to claim 6, wherein the magnetic material comprises Fe₂O₃.

11. The method according to claim 6, wherein the fluorescent dye comprises CdSe or CdSe/ZnS quantum dots.

12. The method according to claim 6, wherein the polymer comprises hydrazinized styrene/acrylamide (H₂N-St-Aam).

13. The nanoparticle according to claim 1, wherein the nanoparticle has no magnetic core.

14. The method according to claim 6, wherein the nanoparticle has no magnetic core.

15. The nanoparticle according to claim 1, wherein the biomaterial is coupled to the nanoparticle with the following structure: 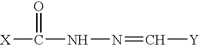
wherein X is the nanoparticle and Y is the biomaterial.

16. The nanoparticle according to claim 15, wherein Y is an antibody, avidin or streptavidin.

17. The method according to claim 6, wherein the biomaterial coupled to the nanoparticle is described by the following formula: 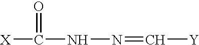
wherein X is the nanoparticle and Y is the biomaterial.

18. The method according to claim 15, wherein Y is an antibody, avidin, streptavidin or biotin.

19. The nanoparticle according to claim 1, wherein the biomaterial is biotin coupled to the nanoparticle with the following structure: 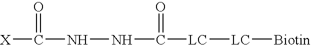
where X is the nanoparticle and LC is —C═O(CH₂)₃—NH—.

20. The method according to claim 6, wherein the biomaterial is biotin coupled to the nanoparticle with the following structure: 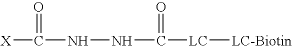
where X is the nanoparticle.

21. A multifunctional nanoparticle, comprising: 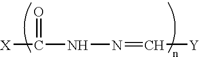
where n≧1;
X is a mesoporous nanoparticle comprising a mesoporous polymer,
a magnetic material adhering to the mesoporous polymer and a fluorescent dye adhering to the mesoporous polymer; and
Y is a protein.

22. The multifunctional nanoparticle of claim 21, in which Y is avidin, streptavidin or an antibody.

23. A multifunctional nanoparticle, comprising: 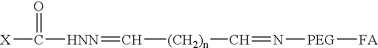
where n≧1;
X is a mesoporous nanoparticle comprising a mesoporous polymer, a magnetic material adhering to the mesoporous polymer and a fluorescent dye adhering to the mesoporous polymer;
PEG is polyethylene glycol; and
FA is folic acid.

24. The multifunctional nanoparticle of claim 23, wherein n is 3.

25. A method for separating cells comprising:
contacting a cell bearing a desired receptor with a multi-functional nanoparticle according to claim 1 in which Y is a ligand that specifically binds to the desired receptor to obtain cells bound with multifunctional nanoparticles;
introducing the cells bound with multifunctional nanoparticles into a magnetic field, thereby immobilizing the cells;
removing cells not bound with multifunctional nanoparticles;
removing the magnetic field from the cells bound with multifunctional nanoparticles, and collecting the cells.

26. A method for separating and sorting cells having different surface receptors comprising:
contacting a sample of cells bearing a plurality of desired receptors with a plurality of multi-functional nanoparticles according to claim 1, in which each kind of multi-functional nanoparticle has a different ligand Y that specifically binds to a desired surface receptor on at least one of said cells in the sample and further in which each ligand Y is paired with a fluorescent dye of a particular color, to obtain cells bound with multifunctional nanoparticles;
introducing the cells bound with multifunctional nanoparticles into a magnetic field, thereby immobilizing the cells;
removing cells not bound with multifunctional nanoparticles;
removing the magnetic field from the cells bound with multifunctional nanoparticles, and collecting the cells;
sorting the collected cells according to the fluorescence color of the dye paired with each ligand Y.

27. A method for isolating and/or detecting biomolecules, comprising:
contacting a biomixture containing a biomolecule bearing a desired interacting site with a multi-functional nanoparticle according to claim 1 in which Y comprises a ligand that specifically binds to a desired receptor or other binding partner to obtain biomolecules bound with multifunctional nanoparticles;
introducing the biomolecules bound with multifunctional nanoparticles into a magnetic field, thereby immobilizing the multifunctional nanoparticles and bound biomolecules;
removing any molecules not bound with the multifunctional nanoparticles;
removing the magnetic field from the biomolecules bound with multifunctional nanoparticles; and
collecting the bound biomolecules.

28. The method of claim 27, which further comprises:
further purifying the bound biomolecules and associated molecules via the fluorescence of the biomolecules bound with the multifunctional nanoparticles.

29. The method of claim 27, wherein the step of further purifying the bound biomolecules occurs either before or after the step of collecting the bound biomolecules.

30. The method of claim 27, wherein the biomolecule comprises a protein.
